(19) United States
(12) Patent Application Publication
(10) Pub. No.: US 2010/0044224 A1
(43) Pub. Date: Feb. 25, 2010

(54) BIOLOGICAL FLUID ANALYSIS SYSTEM

(76) Inventor: Ritu Kataky, Crossgate Moor (GB)

Correspondence Address: Zilka-Kotab, PC, P.O. BOX 721120, SAN JOSE, CA, 95172-1120, US

(21) Appl. No.: 12/441,330

(22) PCT Filed: Sep. 12, 2007
(86) PCT No.: PCT/GB07/03477

§ 371 Date: Nov. 2, 2009

(30) Foreign Application Priority Data
Sep. 13, 2006 (GB) 0617983.2

(51) Int. Cl. G01N 27/26; A61B 5/00; G01N 27/333

(52) U.S. Cl. 204/403.13; 204/407; 204/412

(57) ABSTRACT

A system apparatus (101) includes an integrated sensing and separation component (108) for the monitoring and analysis of clinical species present in biological fluids. In the preferred embodiment this is by use of a polyHIPE membrane (109) with a chemically active sol-gel (110), and a plurality of micro-electrode arrays adapted for selectively adjusting sensitivity.

This invention relates to the field of electrochemical sensors for the monitoring and analysis of ionic and redox active species present in fluids such as biological (including clinical) fluids and botanical fluids. In particular, one embodiment of the invention relates to diagnosis of disease states such as cystic fibrosis via the analysis of biological fluids. However, a number of other embodiments are also envisaged including a sensor for detection of urea and electrolytic analytes.

### BACKGROUND OF THE INVENTION

There are a number of areas where biological fluid analysis is of interest. One particular area of interest relates to the diagnosis of cystic fibrosis.

The clinical disease of cystic fibrosis is a progressive multi-organ disorder, principally characterised by suppurative lung disease, that causes glands in the air passages of the lungs to produce abnormally thick, clogging mucus. In addition, cystic fibrosis also affects the pancreas and exocrine sweat glands. This means that sufferers are prone to high electrolyte loss. Currently there is no known cure for cystic fibrosis, therefore early diagnosis to ensure proper treatment is extremely important to improve the quality of life of the sufferer. There is therefore a preference for the testing of neonates as soon as possible after birth.

Analysis of human sweat has become recognised as an effective method for diagnosing a wide range of disease states, and it has become routine over recent years to carry out sweat testing on neonates where there is a concern that the child may have cystic fibrosis. All neonates undergo screening for cystic fibrosis in the first few days of life. Primarily, a heel prick blood specimen is taken to determine levels of immunoreactive trypsinogen (IRT) by immunoassay. Babies with elevated levels of this biological agent will then be referred for further cystic fibrosis diagnosis using current sweat test methods.

The sweat test is based upon the understanding that the concentration of chloride and sodium ions in sweat samples are elevated in cystic fibrosis sufferers.

Traditionally, tests were carried out on neonates using chemical means to induce sweating in a localised area and using a device for collecting the sweat. Sweating was induced by such methods as pillocartine iontophoresis, typically on the arm of the child. The device for collecting sweat took the form of a gauze pad which was capable of absorbing sweat, which was held in position for a period of time (typically 30-45 minutes) before being removed and the collected sweat eluted. Once sufficient sweat was collected, the concentration of chloride and sodium ions could be measured and compared to calibration standards using techniques including flame photometry, atomic absorption spectrometry or ion selective electrodes. The results, in particular elevated sweat chloride concentrations, are particularly relevant and are associated with cystic fibrosis.

There are a number of problems with the traditional test that make it difficult to use, including unreliable methodology, inadequate sweat collection, technical errors and occasionally misinterpretation of the results. In addition, the technical aspects of performing a sweat test are demanding and such errors are more likely to occur in hospital settings that carry out minimal testing. In view of this, it is often avoided on neonates where, in fact, testing is of the most importance.

There is therefore a need for an improved sensor and test method which yields results from a sample relatively quickly and easily, whilst limiting the evaporation of sweat.

One manner of sweat testing which attempts to mitigate the problems of the traditional apparatus and method utilises a chemical sensor that is able to test for total sweat conductivity rather than analysing chloride and sodium ion concentrations. In this improved apparatus, sweat is collected in a coiled tube which is then detached from, for example, the arm of the patient, and the sweat transferred to a testing area. As the total sweat conductivity test only requires a small volume of sweat, prolonged collection periods are avoided. Whilst this is an improvement over earlier sweat tests, the problem of transporting the collected sweat from the collection point to the test point, and the associated risk of evaporation of the sample, remains. Furthermore, due to the complexity of the biological media, there is often also a necessity for preparation of the sweat sample or pre-treatment to remove contaminants before accurate results can be obtained.

Therefore the need for an improved sensor and test method has not been fully addressed.

Sensors are used to obtain information about their environment and many achieve this by converting measurements from the environment directly into electrical signals that can then be read or upon which further calculations can be performed. Chemical sensing is utilised in a wide range of medical, environmental and industrial applications, with sensing species in chemically harsh media such as whole blood, sweat etc. posing particular problems due to interference for example. Many test methods simply use pre-treatment methods to separate a target species from the original sample but, particularly in the case of medical diagnostic uses, this can often lead to prolonged analysis times for results as well as additional expense. Furthermore, this requires removal of samples from the point of care for analysis under laboratory conditions.

Medical diagnostics can work on the principles of electrochemical models.

One electrochemical sensing technique that is used is programmed absorptive stripping voltammetry. Chemical or biochemical species are accumulated on an electrode of a sensor which is then provided with electrical contact and scanned through a voltage or current range. The sensor is calibrated to indicate characteristic voltages at which known species are desorbed from the electrode. At each characteristic voltage, provided a micro-electrode is used, a steady state constant current yields results which can be converted to the concentration of the known species. Variations of this technique have been described which allow for stability in harsh media. A related field is potentiometric ion-selective electrodes (ISE's) which measure ionic activities rather than concentrations of species. This is of particular relevance in the medical field as ionic activity measurements depict the availabilities of an ionic species as opposed to the total concentrations which include bound and ionic species.

The ions can be measured directly or pre-concentrated and voltage changes measured after the ions are stripped by applying a reverse potential.

Screen printing is a method by which electrodes for use in sensors may be fabricated, the electrode being the underlying conductive layer which forms a link with the electrical measuring equipment.

Furthermore, the specificity of the electrode can be enhanced by modifying said electrode by associating it with an agent which is anchored to the electrode and which can reversibly bind to the species to be detected, releasing it at a particular voltage.

The present invention identifies the drawbacks of the conventional techniques and procedures, and proposes a sensor and method of using the same which mitigates one or more of the limitations previously described.

The aims and objects of the invention will become apparent from reading the following description.

### SUMMARY OF THE INVENTION

An improved method of handling a fluid for analysis is provided, in which the sample is taken, processed to selectively identify a target species, and a result is directly obtainable very soon after taking the sample. The fluid can be a biological fluid or a botanical fluid for example. This advantageous procedure is realised by use of a device adapted to collect the sample, process it and report on its status with regard to a target species of interest. The method may use a specific reagent in the device to treat the sample to develop a detectable moiety having an electrical characteristic. A selective membrane may be used to improve the selectivity of the device to separate collection and detection functions upon exposure to collected sample and target moiety.

According to a first aspect of the present invention there is provided a sensor for the detection of one or more target species comprising an analyte monitoring means characterised in that said analyte monitoring means comprises at least one electrode associated with a selectively permeable membrane adapted to substantially simultaneously select which matter is able to pass through said membrane and selectively and reversibly bind a target species.

This provides the advantage that the sensor is able to substantially simultaneously separate out target species from other contaminants for example, and detect the target species, by means of a single chemically modified interface layer. The use of a single layer in turn provides the advantage that the sensor is able to respond with a significantly faster response time due to enhanced diffusion rates, compared to existing multi-layered technologies. Moreover, the sensor has significant commercial benefits in that it is much easier to mass produce.

The sensor may further comprise a sample collection means.

The electrode may be arranged with respect to the membrane such that collected sample cannot be passed to the electrode without first contacting the membrane.

The selectively permeable membrane may be adapted to pass only matter of predefined size.

The selectively permeable membrane may be adapted to selectively pass or retain a specie, a charged moiety, a micelle or a biological/biochemical entity.

The selectively permeable membrane may be adapted to pass only matter of a predefined size and said analyte monitoring means is adapted to selectively pass or retain a particle, a charged moiety, a micelle or a biological/biochemical entity.

The selectively permeable membrane may comprise at least one pore, wherein at least one said pore is at least partially filled with an ion sensing means.

The ion sensing means may be a chemically modified solgel.

The selectively permeable membrane may comprise polyHIPE.

The at least one electrode may comprise a multiplicity of micro-electrode arrays whereby said micro-electrode arrays are configured such that they are selectively connectable to each other in order to alter the number of micro-electrode arrays available for the detection of a particular target species.

This provides the advantage that species of even very low concentrations can be detected in view of the ability of the micro-electrode arrays to significantly enhance the electrical signal. Moreover, more than one species can be detected in real time, and substantially simultaneously, using the multi array technology. For example a single sensor can detect two different species at significantly different concentrations by varying the number of arrays used to detect each species. Thus a first species at high concentration may be detectable using a single array and a second species may be detectable at the same time using the same sensor by using multiple arrays to detect it where it is typically in a sample at a lower concentration than the first species.

The sensor may be disposable.

The analyte monitoring means may be configured to detect ions.

The sample collection means and analyte monitoring means may be in physical contact.

The sample collection means may be gauze adapted to take up biological fluid.

Gauze is particularly useful for collecting sweat for neonates as it can be placed onto an infants arm with minimal discomfort.

The sample collection means may comprise a tube.

Using a tube to collect samples minimises the risk of evaporation of the sample during the collection process as the sample is substantially contained within the tube.

The tube may be in the form of a coiled duct.

The analyte monitoring means may comprise one or more screen printed electrodes.

Screen printed electrodes offer a low cost, disposable highly reproducible alternative to traditional electrodes.

The electrodes may be Ag/AgCl screen printed electrodes modified to act as ion selective electrodes.

The sensor may comprise an internal reference electrode and an external reference electrode with an internal filling layer therebetween.

The internal filling layer may be a chemically modified hydrogel.

The hydrogel may be selected from polyvinyl alcohol (PVA) and polyvinyl pyrrolidone (PVP).

The analyte monitoring means may be adapted to sense one or more analytes selected from the group comprising:

Cholesterol;

Anaesthetics;

cardiac markers;

oncology biomarkers;

urea and electrolyte analytes;

haemoglobin;

glucose;

high density lipases (HDL); and

Low Density Lipases (LDL).

Optionally the selectively permeable membrane is adapted to pass a certain size of particle, e.g. a molecular weight cut-off (MWCO) limited range. Alternatively, or in addition, it may be adapted to pass material according to surface effect or differences in electrophonetic mobilities. This ability to speciate in situ is very useful for process control.

The membrane may be adapted to selectively pass or retain a particle, a charged moiety, a micelle or a biological/biochemical entity. The entity may be an amino acid, a peptide, protein, a lipoprotein, a hormone, an enzyme, a viral particle or prion, a biological marker or antigen, an antibody or a fragment thereof, a lipid, a fatty acid, a carbohydrate, a trace element, a metabolite, or a cellular excreted component.

The analyte monitoring means may be in the form of a planar strip.

According to a second aspect of the present invention there is provided a sensor for detection of at least one target species, the sensor comprising:

at least one membrane having at least one at least partially porous material adapted to selectively allow permeation of some species into said membrane and having at least one detection medium adapted to produce at least one signal in response to the presence of at least one target species, the or each detection medium being at least partially contained within pores in said porous material; and

at least one electrode for passing at least one said signal to a signal analysing machine.

According to a third aspect of the present invention there is provided a device comprising a sensor unit for detection of a target species which may be present in fluids, comprising a sample collection means adapted to collect a fluid, together with an analyte monitoring means configured to receive and detect an electrical characteristic of a moiety derived from the association of a reagent capable of selectively and reversibly associating with the target species, which characteristic is distinguishable from electrical characteristics in the absence of such association, and a selectively permeable membrane,

wherein the sample collection means and analyte monitoring means are proximal to each other to allow substantially simultaneous collection and monitoring of the fluid.

According to a fourth aspect of the present invention there is provided a sensor unit for detection of analytes present in fluids comprising a sample collection means adapted to collect a fluid and an analyte monitoring means wherein the analyte monitoring means comprises an electrode associated with a modifying agent able to selectively and reversibly bind to a species of interest, and a porous membrane adapted to select the particles which are able to pass through it, and wherein the sample collection means and analyte monitoring means are adapted to allow substantially simultaneous collection and monitoring of the fluid.

The porous membrane may be a size selective polymer membrane.

The porous membrane may comprise polyHIPE.

According to a fifth aspect of the present invention there is provided a device for the detection of one or more target species comprising a multiplicity of microelectrode arrays whereby said microelectrode arrays are configured such that they are selectively connectable to each other in order to alter the number of micro-electrode arrays available for the detection of a particular target species.

The device may further comprise the sensor as previously disclosed.

The incorporation of both an analyte monitoring means and a sample collection means into a single sensor unit means that the results from the sensor can be obtained at the point of collection, thus providing a rapid, cost effective solution to the aforementioned problems. Furthermore, providing the clinical species sensing means in a form incorporating a porous membrane ensures that the sensor can be used directly on the sample without the need for substantial pre-treatment to isolate the analyte or analytes of interest as the sensor is providing both sensing and separation capabilities.

In order to provide a better understanding of the present invention, embodiments will be described by way of example only and with reference to the following figures;

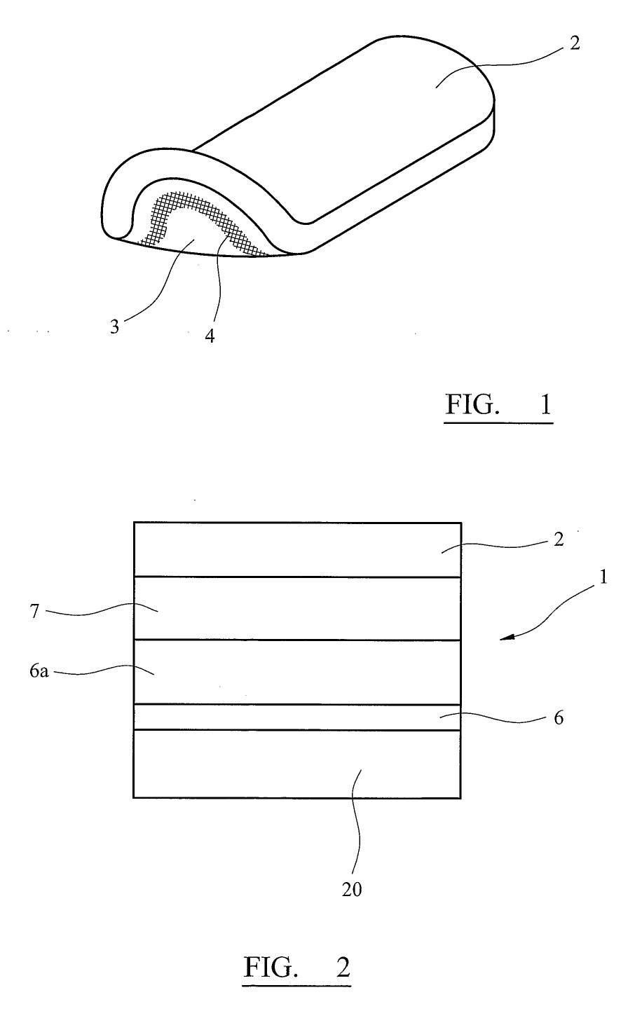

FIG. 1 is a diagram of a portion of an apparatus according to a first embodiment of the present invention, suitable for example, for use in the diagnosis of cystic fibrosis in neonates; and

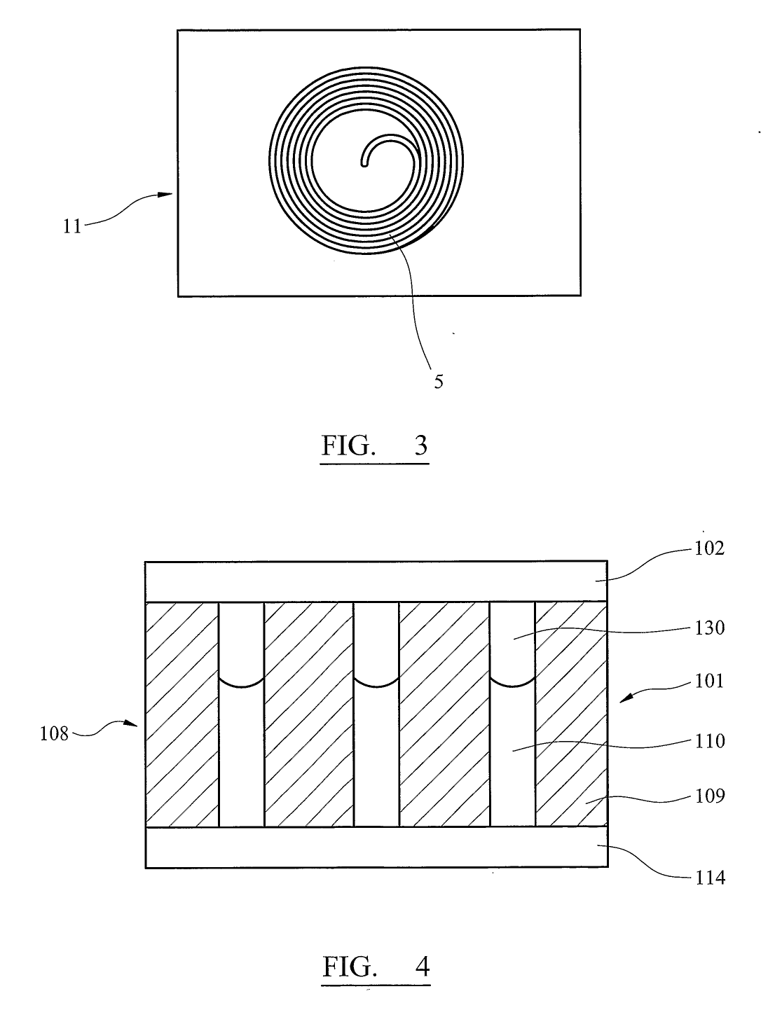

FIG. 2 is a diagram of an apparatus according to a first embodiment of the present invention; and

FIG. 3 is a diagram of an apparatus according to a second embodiment of the present invention;

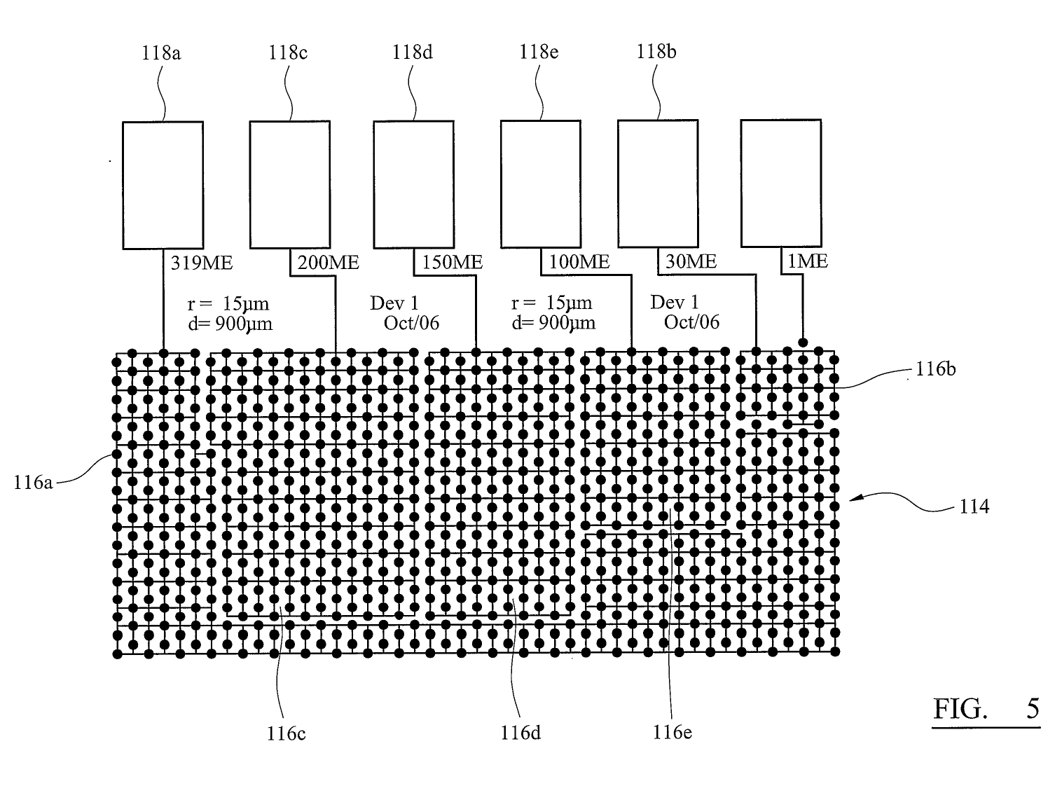

FIG. 4 is a diagram of an apparatus according to a third, preferred, embodiment of the present invention;

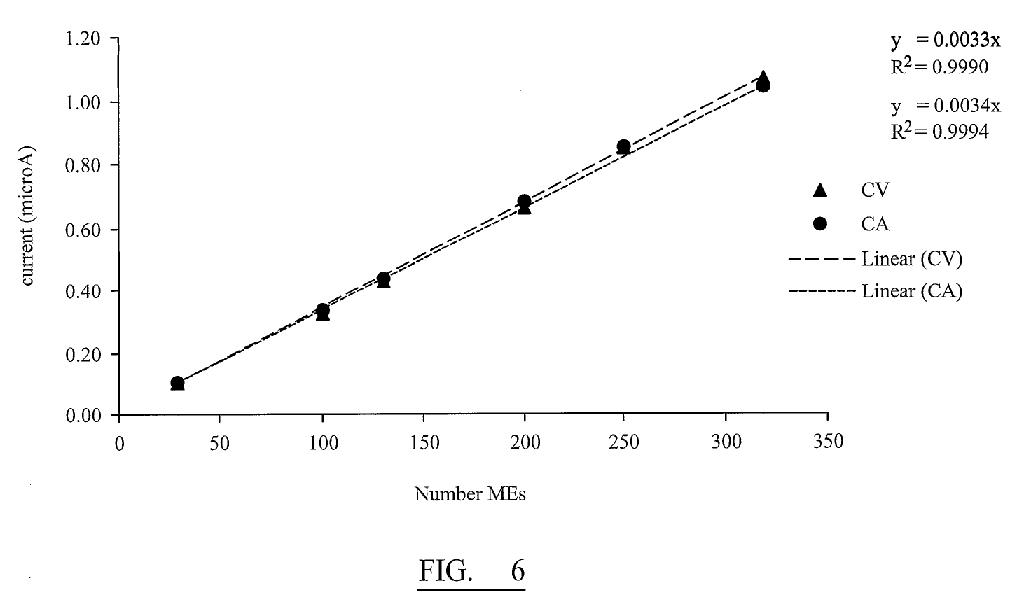

FIG. 5 is a diagram of a plurality of micro-electrode arrays forming part of the apparatus of FIG. 4; and

FIG. 6 is a plot of the current response related to the number of micro-electrodes connected to a single electrical contact pad.

The examples are described with particular reference to the diagnosis of cystic fibrosis in neonates by the testing of sweat. However, it is to be appreciated that this is merely illustrative and that the apparatus described can be adapted for use in the detection of a wide range of clinical species associated with either disease states, blood-drug concentrations or substance misuse. For example, the apparatus can be used to detect sodium ions, potassium ions, calcium ions, magnesium ions and chloride. Moreover, the apparatus can provide an indication of pH, and can detect lactate, glucose, ascorbate and 3-Hydroxy-3 methylhexanoic acids (which are responsible for sweat odours). Further, the apparatus can be used in the detection of schizophrenia, exercise stress related conditions and in the deodorant industry.

In particular it is envisaged that the sensor of the present invention could be used to provide a point of care (POC) test kit for use in hospital departments. POC test kits allow clinical analysis to be carried out at the patient's bedside thus avoiding delays associated with standard laboratory analysis. This is particularly beneficial for samples such as arterial blood samples which are particularly prone to pre-test variables such as temperature and atmosphere which can effect analysis and test results. Primarily such kits will be developed for use in Accident and Emergency units to provide an immediate indication of a patient's status and renal function following the rapid determination of sodium, potassium, chloride, urea, bicarbonate, glucose and haemoglobin levels in whole blood samples.

With particular reference to FIGS. 1 and 2, in a first embodiment of the present invention there is provided sweat testing apparatus 1. The apparatus 1 comprises a gauze pad 2 adapted to be positioned on the arm of an infant such that the anterior surface 3 is in contact with the infant's skin. The gauze pad 2 is adapted to take up sweat from the infant's skin and is provided with an adhesive 4 on the anterior surface 3 to hold it in position. Alternative embodiments can be envisaged where adhesive strips are placed over the posterior surface of the gauze pad 2 and extending onto the infant's arm, or where a bandage can be wrapped around the infant's arm encasing or overlapping the gauze pad 2.

The posterior surface of the gauze pad 2 is provided with an ion sensing layer 6 as can be seen in FIG. 2. In the present invention, the ion sensing layer 6 is provided with chloride and sodium ion sensitive inks being screen printed onto a proton electron transfer (PET) substrate with a polyHIPE membrane overlay 7. The apparatus 1 further comprises an electrode 20 on the opposite side of the ion sensing layer 6 to the polyHIPE membrane overlay 7.

In this embodiment of the device, the sensor part of the apparatus is composed of 3 integrated components. The first component 6 is for sensing; the second component 6a for chemically breaking down interferrants, for example, proteins in whole blood; and the third component 7 a membrane of controllable porosity to assist in the filtration of particulate matter. The sensing layer 6 comprises a chemical or biological ligand physically or chemically immobilised on a polymer. The polymer may be PVC(poly vinyl chloride), poly urethane, or polyHIPE. The polymers may contain conducting particles such as activated carbon, gold, platinum or silver. The second component 6a may be cellulose acetate or PolyHIPE containing reagents to break down interrferants. The third component 7 is PolyHIPE, a polymer with pores of tuneable porosity to assist in the filtration of particulates such as blood corpuscles and grit. It is however to be appreciated that other porous or selectively permeable materials such as cellulose acetate and collagen could be used instead of or in addition to the polyHIPE interfaces described above.

As one of the embodiments of the present invention is for use in the diagnosis of cystic fibrosis, both a chloride sensing ink and a sodium ion sensing ink are used in the electrode printing process, although more simplistic variations can be produced using only one of the inks. In one embodiment Ag/AgCl screen printed electrodes consist of an Ag/AgCl internal reference electrode and an Ag/AgCl external reference electrode. These were then modified to work as ion selective electrodes (ISEs). When preparing the sodium ISE, hydrogel material (e.g. polyvinyl alcohol (PVA), polyethyleneglycol (PEG) or polyvinyl pyrrolidone (PVP)) is dissolved in 10⁻³ M sodium chloride solution and layered onto the internal reference electrode. The electrode was then allowed to dry for 8 hours in an environment substantially free from dust and other particulate matter.

The polyHIPE interfaces are prepared from polymerisation in a high internal phase emulsion (HIPE) template. Water is emulsified in a mixture of hydrophobic monomer, cross-linker and surfactant. The resulting HIPE is cured causing the monomers to polymerise around the emulsion droplets resulting in a porous material. Due to volume contraction upon polymerisation, small pores or interconnects open up between adjacent water droplets. The water can then be removed, yielding a porous, permeable, low density material. Ligands and other chemical entities capable of sensing charged species e.g. enzymes, graphite particles, or activated carbon fibres, can then be incorporated.

Commercially available ligands and ligands that can be synthesised according to the laboratory methods described in “Comparative Study of Tripodal Oxamide and Oxaesters as lonophores in Potentiometric Ion-Selective Electrodes for Alkali and Alkaline Earth Cations;” R. Kataky, D. Parker, A Teasdale; Anal Chim Acta; 276; 353-360, 1993, can be used when measuring chloride and sodium ions.

A silver/silver chloride (Ag/AgCl) solid state electrode may also be used to measure chloride ions. An alternative to a polyHIPE (polymer formed in a high internal phase emulsion) membrane can be envisaged, whereby a sodium ion selective PVC hydrogel layer is used.

In an alternative embodiment of the invention, as generally depicted in FIG. 3, the sweat testing apparatus 11 is provided with a coiled duct 5 in the form of a tube. The coiled duct is part of the collection apparatus which collects sweat within it. A screen printed electrode is disposed within the coiled duct such that it is able to contact the sweat sample providing a reading at substantially the same time as the collection occurs. If required the collected sample can then be utilised for further analysis.

In a preferred embodiment of the invention, as generally depicted with reference to FIGS. 4 to 6, there is provided an apparatus 101 for detecting a target species in a biological fluid.

The apparatus 101 comprises a gauze pad 102 which is adapted to take up biological fluid for example, by capillary action. A first surface of the gauze pad 102 is provided with an analyte monitoring means in the form of an ion sensing layer 108. The ion sensing layer 108 comprises a polyHIPE membrane 109 which includes a plurality of small interconnected pores 130 dispersed over the surface thereof. In this way, the polyHIPE membrane 109 is a porous, permeable and low density material, and may be made in a similar fashion to the porous polyHIPE interfaces described with reference to FIGS. 1 and 2. The ion sensing layer 108 further comprises a quantity of solgel 110 made in a room temperature ionic liquid. In particular, the pores 130 in the membrane 109 are partially filled with solgel 110 using capillary action. The solgel 110 includes chemical entities such as ligands or ionophores (not shown in the Figures) which result in the ion sensing layer 108 becoming chemically active.

The solgel 110 further includes a plurality of carbon nanotubes (not shown on the Figures) which provide an increased surface area to thereby assist in catalysis when required.

The ion sensing layer 108 may be of any suitable thickness. However, the inventor has found that a layer 108 having a thickness of approximately 500 microns is particularly convenient.

The solgel 110 further includes enzymes such as pepsin which can break down proteins.

The solgel used in this embodiment is polyvinyl acetate (PVA), but it is to be appreciated that other solgels such as polyvinyl pyrrolidone (PVP) could alternatively be used.

In this way, the ion sensing layer 108 is chemically active and performs both the separating step and the sensing step. Moreover, the apparatus 101 is able to carry out the separating and sensing steps substantially simultaneously. In particular, the sensing layer 108 is able to separate particulate matter from the analyte using the polyHIPE membrane 109 and at the same time, sense the charged species using the chemically active solgel 110.

The ion sensing layer 108 further comprises a plurality 114 of micro-electrode arrays, whereby the micro-electrode arrays may be selectively connected and disconnected in order to increase or decrease the sensitivity of the apparatus, according to the concentration of analyte in the biological fluid. A schematic Figure of the micro-electrode arrays is shown in FIG. 5.

As can be seen from FIG. 5, each of a plurality of micro-electrode arrays 116a to 116e represent an interconnected network of micro-electrodes, such that each micro-electrode array is connected to a particular contact pad 118a to 118e. In particular, a first micro-electrode array 116a is linked to a first contact pad 118a, a second micro-electrode array 116b (containing a different number of micro-electrodes) is linked to a second contact pad 118b, and so on until each of the contact pads are electrically connected to an array of micro-electrodes.

The electrodes may be screen printed or produced by photolithography techniques. The first contact pad 118a can for example, be used in the detection of a predetermined analyte, the second contact pad 118b for the detection of a different analyte, and so on. This is particularly useful as this offers a low cost, disposable, highly reproducible option which can easily be modified depending on the type of sensor required. Multiple arrays of micro-electrodes linked to different electronic pathways allow differentiation and recognition of multiple analytes on a single device. The multiplexing of the micro-electrodes allows signals from analytes with low concentrations to be intensified.

An example where the multiplexing of electrode signals can be used to intensify signals from low concentration analytes can be seen in the electrochemical analysis of calcium and sodium ions in whole blood where concentrations are 2.3 mmolL⁻¹ and 150 mmolL⁻¹ respectively. A greater number of micro-electrodes are utilised for calcium detection than for sodium detection as the calcium concentrations are lower. The multiplexed electrode signals for calcium allow the signal to be enhanced without compromising the signal for other analytes such as sodium. Again FIG. 5 shows an example where different numbers of microelectrodes may be used depending on the concentration of the analytes.

FIG. 6 is a plot of the current response related to the number of micro-electrodes connected to a single electrical contact pad, which could for example be a gold pad. As can be seen from this Figure, the current sensitivity increases linearly with the number of micro-electrodes electrically connected to each contact pad. In order to detect a target species which has a low concentration in the fluid being analysed, a greater number of micro-electrodes is required to be connected in comparison to the situation whereby the target species has a high concentration in the fluid being analysed.

In order to use the apparatus 101, the user first dips the apparatus 101 into the analyte (or otherwise exposes the gauze pad 102 and thereby the ion sensing layer 108, to the analyte). Some of the particulate matter which may be present does not pass from the gauze pad 102 into the pores of the polyHIPE membrane 109, and is instead held outside of the pores. The remaining biological fluid then passes into the pores of the polyHIPE membrane 109 which themselves are partially filled with the solgel 110.

The solgel 110 is itself conductive and is electrically connected to the micro-electrodes.

The targeted species is selectively recognised by the sensing elements (such as ligands for example) in the solgel 110, which provide a characteristic change in the voltage or the current.

The apparatus of the present invention provides many benefits. It can provide a soft, biocompatible sensor which is low cost and disposable, using ion-selective electrodes which are easy to use at the bedside and can be mass produced.

The selectivity and sensitivity of the sensor is greatly increased through the modification of an electrode with a chemical or physical agent. The fact that the analyte monitoring means selectively and reversibly binds the species of interest means that it controls the mobilisation and transport of said species thus inherently forming a separation as well as a sensing function.

The present invention in one of its aspects provides an improved apparatus for testing the sweat of infants for analyte levels suggestive of cystic fibrosis. Whilst this apparatus is particularly useful for neonates it could easily be used on adults as well. It can also be seen that the apparatus can be adapted to sense a wide range of analytes indicative of a range of disease states or body conditions (such as hyponatremia, hypernatremia) and allows determination of electrolyte balance. As mentioned previously, one particular area where the invention can be utilised is in POC test kits for use in hospital departments and GP clinics to provide rapid determination of a patient's status and renal function.

Yet further embodiments can be envisaged where the apparatus is not designed to collect sweat but is instead adapted to collect and monitor other bodily fluids. This could include apparatus for collection of blood or skin samples, or could take the form of a breathalyser type apparatus which would be particularly useful in identifying substance misuse. It can also be envisaged that the sensor apparatus can be used for enzyme-based sensing, immunosensing, whole cell-based sensing, nucleic acid-based sensing and chemical sensing.

The present invention relates to a platform technology that can be chemically modified with biological entities to in theory detect any biological component should the antibodies or laboratory synthesised chemical entities be available.

The apparatus is capable of analysing several related, chemically distinct analyte species simultaneously, in real time and directly. Analytes in sweat such as sodium ions, potassium ions, calcium ions, magnesium ions, chloride, pH, lactate, glucose, ascorbate, 3-Hydroxy-3 methylhexanoic acids (responsible for sweat odours) may be analysed simultaneously yet distinctly. The apparatus may be modified with chemically active, biocompatible, porous materials such as cellulose, collagen and polyHIPEs which filter out particulate matter and break down possible interferrants such as protinaceous material.

It is to be appreciated that the apparatus may be used for applications other than cystic fibrosis detection, such as detection of schizophrenia, exercise stress related conditions and in the deodorant industry my modifying each group of micro-electrodes with selective agents such as ionophores and enzymes.

It will be appreciated by persons skilled in the art that the above embodiment has been described by way of example only, and not in any limitative sense, and that various alterations and modifications are possible without departure from the scope of the invention as defined by the appended claims.

1-33. (canceled)

34. A sensor device for the detection of one or more target species comprising an analyte monitoring means, said analyte monitoring means comprising a multiplicity of microelectrode arrays whereby said microelectrode arrays are configured such that they are selectively connectable to each other in order to alter the number of micro-electrode arrays available for the detection of a particular target species.

35. A sensor device as claimed in claim 34, wherein said analyte monitoring means, further comprises a selectively permeable membrane associated with at least one said microelectrode array, said selectively permeable membrane being adapted to substantially simultaneously select which matter is able to pass through said membrane and selectively and reversibly bind a target species.

36. A sensor device as claimed in claim 34, further comprising a sample collection means.

37. A sensor device as claimed in claim 36, wherein the electrode is arranged with respect to the membrane such that collected sample cannot be passed to the electrode without first contacting the membrane.

38. A sensor device as claimed in claim 35, wherein the selectively permeable membrane is adapted to pass only matter of predefined size.

39. A sensor device as claimed in claim 35, wherein the selectively permeable membrane is adapted to selectively pass or retain a species, a charged moiety, a micelle or a biological/biochemical entity.

40. A sensor device as claimed in claim 35, wherein the selectively permeable membrane comprises at least one pore, wherein at least one said pore is at least partially filled with an ion sensing means.

41. A sensor device as claimed in claim 40, wherein the ion sensing means is a chemically modified solgel.

42. A sensor device as claimed in any one of the previous claims wherein the analyte monitoring means is configured to detect ions.

43. A sensor device as claimed in claim 36, wherein the sample collection means and analyte monitoring means are in physical contact to allow substantially simultaneous collection and monitoring of the fluid.

44. A sensor device as claimed in claim 36, wherein the sample collection means is gauze adapted to lake up biological fluid.

45. A sensor device as claimed in claim 34, wherein the analyte monitoring means comprises one or more screen printed electrodes.

46. A sensor device as claimed in claim 45, wherein the electrodes are Ag/AgCl screen printed electrodes modified to act as ion selective electrodes.

47. A sensor device as claimed in claim 34, comprising an internal reference electrode and an external reference electrode with an internal filling layer therebetween.

48. A sensor device as claimed in claim 47, wherein the internal filling layer is a chemically modified hydrogel.

49. A sensor device as claimed in claim 48, wherein the hydrogel is selected from polyvinyl alcohol (PVA) and polyvinyl pyrrolidone (PVP).

50. A sensor device as claimed in claim 35, wherein said selectively permeable membrane is adapted to produce at least one signal in response to the presence of at least one target species, whereby at least one said electrode is adapted to pass at least one said signal to a signal analysing machine.

51. A sensor device as claimed in claim 34, wherein said sensor device is configured to receive and detect an electrical characteristic of a moiety derived from the association of a reagent capable of selectively and reversibly associating with the target species, which characteristic is distinguishable from electrical characteristics in the absence of such association.

52. A sensor device as claimed in claim 35, wherein the selectively permeable membrane is a size selective polymer membrane.

53. A sensor for the detection of one or more target species comprising at least one analyte monitoring device characterised in that said at least one analyte monitoring device comprises at least one sensor device according to claim 34, the sensor device associated with a selectively permeable membrane adapted to substantially simultaneously select which matter is able to pass through said membrane and selectively and reversibly bind a target species.
